(19) United States
(12) Patent Application Publication
(10) Pub. No.: US 2001/0006778 A1
(43) Pub. Date: Jul. 5, 2001

(54) Methods for identifying compounds that bind to a target

(75) Inventors: Howard Benjamin, Lexington, MA (US); Mark A. Findeis, Cambridge, MA (US); Malcolm L. Gefter, Lincoln, MA (US); Gary Musso, Hopkinton, MA (US); Ethan R. Signer, Cambridge, MA (US)

Correspondence Address: LAHIVE & COCKFIELD, 28 STATE STREET, BOSTON, MA, 02109, US

(73) Assignee: Praecis Pharmaceuticals Incorporated

(21) Appl. No.: 09/772,116

(22) Filed: Jan. 26, 2001

Related U.S. Application Data
(63) Continuation of application No. 08/573,786, filed on Dec. 18, 1995, now abandoned.

(51) Int. Cl. C12P 21/06; C12Q 1/68

(52) U.S. Cl. 435/5; 435/6; 435/7.1; 435/69.1

(57) ABSTRACT

Methods for identifying a compound that binds to a target are described. In general, the methods involve forming a first library comprising a multiplicity of peptides, identifying one or more peptides that bind to the target and determining a peptide motif therefrom, forming a second library comprising a multiplicity of compounds designed based on the peptide motif, selecting from the second library at least one compound that binds to the target, and determining the structure or structures of the at least one compound that binds to the target. Libraries of compounds based on a peptide motif and compounds identified by the methods of the invention are also disclosed.

### BACKGROUND OF THE INVENTION

Recent advances in methods for producing large libraries of peptides have provided unprecedented numbers of peptides which can be screened for pharmaceutical activity. Both chemical and biological methods for synthesis of peptide libraries have been reported. For example, libraries of peptides (e.g., having 10⁶-10¹² member peptides) can be displayed on the surface of bacteriophage (known as “phage display” libraries). Such peptide libraries can comprise all possible peptides of a given length (e.g., every one of the twenty natural amino acid residues at each position of a hexamer), or a subset of all possible peptides. Methods for screening large libraries of peptides, to identify those peptides that bind to a target, have also been developed, such as biopanning. These screening techniques allow for the isolation from a library of one, or several, peptides that bind to a pre-selected target. By producing and screening large peptide libraries, it has become possible to rapidly search for peptides (e.g., ligands) that bind to a target (e.g., a receptor). Moreover, the structure of selected peptides can be determined with relative ease by standard sequencing methodologies (e.g., sequencing of the peptides themselves or of a nucleic acid molecule encoding the peptide).

Despite the advantages of peptide libraries (e.g., immense diversity and simple “deconvolution” of the peptide structure by sequencing), the use of this approach to identify peptides that bind a target for pharmaceutical purposes has a number of drawbacks. For example, the affinity of a selected peptide(s) for the target often is relatively low (e.g., high enough to detect binding of the peptide to the target but too low for pharmaceutical potency). Moreover, peptides are not always suitable for therapeutic administration due to such problems as difficulties in formulation (due to insolubility), unfavorable pharmacokinetics and/or pharmacodynamics, and rapid degradation in vivo.

Alternative to peptide libraries, libraries of non-peptide chemical compounds (e.g., peptidomnimetics, peptide derivatives, peptide analogues, etc.) can be synthesized. Screening of a target with a non-peptide library may lead to the identification of a compound(s) with higher affinity for the target than that of a peptide selected by random peptide library screening and/or identification of a compound(s) with more desirable pharmacological properties than a peptide. However, the diversity of compounds that can be achieved by random chemical synthesis is considerably lower than that of random peptide library synthesis, thereby reducing the likelihood of identifying a high affinity target-binding compound from a randomly synthesized chemical library. An additional disadvantage of a chemical library approach to identifying molecules that bind a target is that determination of the structure of the compound(s) that binds the target (i.e., “deconvolution” of the compound structure) cannot be accomplished by a simple sequencing methodology but rather requires more complex chemical strategies, thereby limiting the number of identified compounds that can be efficiently analyzed.

Improved methods for identifying compounds that bind a target that retain the advantageous properties of both peptide library screening and chemical library screening while reducing or eliminating the disadvantageous properties of these techniques are needed.

### SUMMARY OF THE INVENTION

The present invention features methods for identifying compounds that bind a target that combine the use of peptide-based libraries with the use of chemically-based libraries such that the advantages of each approach are maintained while many of the disadvantages of using either approach alone are overcome. For example, the methods of the invention provide the diversity and ease of deconvolution of traditional peptide library screening yet also provide for the identification of compounds with high affinity for the target and desirable pharmacological properties. To optimize the benefits of both peptide-based and chemically-based libraries, the methods of the invention involve utilizing information obtained from screening a target with a first library comprising a multiplicity of peptides in the design of a second library comprising a multiplicity of chemical (i.e., non-peptide) compounds. The target is then rescreened with this second library to identify compounds that bind to the target.

The methods of the invention generally involve the following steps:

a) forming a first library comprising a multiplicity of peptides;

b) selecting from the first library at least one peptide that binds to the target;

c) determining the sequence or sequences of the at least one peptide that binds to the target, thereby forming a peptide motif;

d) forming a second library comprising a multiplicity of non-peptide compounds designed based on the peptide motif;

e) selecting from the second library at least one non-peptide compound that binds to the target; and

f) determining the structure or structures of the at least one non-peptide compound that binds to the target;

thereby identifying a compound that binds to the target.

The first library is composed of peptides whose structures can be determined by standard sequencing methodologies (e.g., direct sequencing of the amino acids making up the peptides or sequencing of nucleic acid molecules encoding the peptide). Thus, the first library provides the extensive diversity of peptide libraries and the ease of deconvoluting the selected peptides. In contrast, the second, non-peptide library preferably comprises compounds that, while not peptides, are structurally related to peptides, such as peptide analogues, peptide derivatives and/or peptidomimetics. The structure of the non-peptide compounds preferably is determined by a mass spectrometric method, most preferably by tandem mass spectrometry. Since the second library is designed based on the peptide motif generated from screening the first library, many of the disadvantages of traditional chemical libraries (such as reduced diversity and more laborious deconvolution methods) are reduced or eliminated, since the second library is “biased” toward compounds that have affinity for the target. This bias in the second library for compounds having affinity for the target means that fewer compounds need to be screened as compared to a random chemically-synthesized library and, accordingly, fewer compounds need to be analyzed structurally (i.e., deconvoluted).

The methods of the invention can further involve additional library screening steps. For example, after compounds from the second library that bind the target have been identified, a third library can be formed that comprises a multiplicity of non-peptide compounds designed based on the structure or structures of the non-peptide compounds identified from the second library. The target can be rescreened with the third library to identify additional compounds that binds to the target.

Another aspect of the invention pertains to a library comprising a multiplicity of non-peptide compounds designed based on a peptide motif, wherein the peptide motif is determined by selecting from a peptide library at least one peptide that binds to a target, determining the sequence or sequences of the at least one peptide that binds to the target and determining a peptide motif.

Yet another aspect of the invention pertains to compounds identified by a method the invention. In a preferred embodiment, the compound is a peptidomimetic. In other preferred embodiments, the compound that binds to a target has a binding affinity for the target of at least about 10⁻⁷ M, more preferably at least about 10⁻⁸ M, and even more preferably at least about 10⁻⁹ M.

### DETAILED DESCRIPTION OF THE INVENTION

The present invention pertains to methods for identifying a compound that binds to a target, as well compounds identified thereby, and libraries for use in the methods of the invention. The methods of the invention involve screening a target with at least two distinct libraries. The term “target”, as used herein, is intended to include molecules or molecular complexes with which compounds (e.g., peptides or non-peptide compounds) can bind or interact. Exemplary targets include ligands, receptors, hormones, cytokines, antibodies, antigens, enzymes, and the like. The target can be, for example, a purified compound or a partially purified compound or it can be associated with the surface of a cell that expresses the target.

In the methods of the invention, a target is initially screened with a peptide library to generate a peptide motif for peptides that can bind to the target. Accordingly, the methods of the invention first involve:

forming a first library comprising a multiplicity of peptides;

selecting from the first library at least one peptide that binds to the target; and

determining the sequence or sequences of the at least one peptide that binds to the target, thereby generating a peptide motif.

The term “peptides”, as used herein with regard to libraries, is intended to include molecules comprised only of natural amino acid residues (i.e., alanine, arginine, aspartic acid, asparagine, cysteine, glutamic acid, glutamine, glycine, histidine, isoleucine, leucine, lysine, methionine, phenylalanine, proline, serine, threonine, tryptophan, tyrosine and valine) linked by peptide bonds, or other residues whose structures can be determined by standard sequencing methodologies (e.g., direct sequencing of the amino acids making up the peptides or sequencing of nucleic acid molecules encoding the peptide). The term “peptide” is not intended to include molecules structurally related to peptides, such as peptide derivatives, peptide analogues or peptidomimetics, whose structures cannot be determined by standard sequencing methodologies but rather must be determined by more complex chemical strategies, such as mass spectrometric methods.

The term “multiplicity”, as used herein, refers to a plurality of different molecules (e.g., peptides or non-peptide compounds). Thus a “library comprising a multiplicity of peptides” refers to a library of peptides comprising at least two different peptide members. In preferred embodiments, libraries of peptides useful in the present invention include at least about 10³ different peptides, more preferably at least about 10⁶ different peptides and even more preferably at least about 10⁹ different peptides. Depending on the length of the peptide members and the efficiency of synthesis, library diversity as high as about 10¹² different peptides or even about 10¹⁵ different peptides may be achievable. A library comprising a multiplicity of peptides for use in the methods of the invention can comprise all possible peptides of a specified length (i.e., a “complete” random library wherein each position of the peptide can be any one of the twenty natural amino acid residues, e.g., all possible hexapeptides). Alternatively, a peptide library can include only a subset of all possible peptides of a specified length by having non-degenerate positions within the peptide library (i.e., one or more positions within the peptide which are occupied by only one, or a few, different amino acid residue(s) within each peptide member of the library). Moreover, as the peptide length increases, it may not be possible to achieve every possible peptide permutation within the library. Preferably, at least about 10⁵ to 10⁸ permutations of all possible permutations of a randomized peptide are present within the library. The length of the peptides used in the library can vary depending upon, for example, the degree of diversity desired and the particular target to be screened. For example, in different embodiments, the peptide library is made up of peptides not longer than about 30 amino acids long, not longer than about 20 amino acids long or not longer than about 12 amino acids long. Preferably, the peptide library is comprised of peptides at least 3 amino acids long, and more preferably at least 6 amino acids long.

A library comprising a multiplicity of peptides can be formed by any one of several methods known in the art. For example, in one embodiment, a multiplicity of nucleic acid molecules encoding a multiplicity of random peptides are synthesized and the nucleic acid molecules are introduced into a vector that allows for expression of the encoded peptide library. One examples of such a library is an “external” library in which the peptide library is expressed on a surface protein of a host, such as a “phage display” library (see, e.g., Smith, G. P. (1985) Science 228:1315-1317; Parmley, S. F. and Smith, G. P. (1988) Gene 73:305-318; and Cwirla, S. et al. (1990) Proc. Natl. Acad. Sci. USA 87:6378-6382). As used herein, a “phage display” library is intended to refer to a library in which a multiplicity of peptides is displayed on the surface of a bacteriophage, such as a filamentous phage, preferably by fusion to a coat protein of the phage (e.g., the pill protein or pVIII protein of filamentous phage). In phage-display methods, a multiplicity of nucleic acid molecules coding for peptides is synthesized and inserted into a phage vector to provide a recombinant vector. Suitable vectors for construction of phage display libraries include fuSE vectors, such as fUSE1, fUSE2, fUSE3 and fUSE5 (Smith and Scott (1993) Methods Enzymol. 217:228-257). Nucleic acid molecules can be synthesized according to methods known in the art (see, e.g., Cormack and Struhl, (1993) Science 262:244-248), including automated oligonucleotide synthesis . Following insertion of the nucleic acid molecules into the phage vector, the vector is introduced into a suitable host cell and the recombinant phage are expressed on the cell surface after a growth period. The recombinant phage can then be used in screening assays with a target (described further below).

Another example of a peptide library encoded by a multiplicity of nucleic acid molecules is an “internal” library, wherein the peptide members are expressed as fusions with an internal protein of a host (i.e., a non-surface protein) by inserting the nucleic acid molecules encoding the peptides into a gene encoding the internal protein. The internal protein may remain intracellular or may be secreted by, or recovered from, the host. Examples of internal proteins with which peptide library members can be fused include thioredoxin, staphnuclease, lac repressor (LacI), GAL4 and antibodies. An internal library vector is preferably a plasmid vector. In one example of an internal library, referred to as a two-hybrid system (see e.g., U.S. Pat. No. 5,283,173 by Field; Zervos et al. (1993) Cell 72:223-232; Madura et al. (1993) J. Biol. Chem. 268:12046-12054; Bartel et al. (1993) Biotechniques 14:920-924; and Iwabuchi et al. (1993) Oncogene 8:1693-1696), nucleic acid molecules encoding a multiplicity of peptides are inserted into a plasmid encoding the DNA binding domain of GAL4 (GAL4db) such that a library of GAL4db-peptide fusion proteins are encoded by the plasmid. Yeast cells (e.g., Saccharomyces cerevisiae YPB2 cells) are transformed simultaneously with the plasmid encoding the library of GAL4db-peptide fusion proteins and a second plasmid encoding a fusion protein composed of the target fused to the activation domain of GAL4 (GAL4ad). When the GAL4ad-target interacts with a GAL4db-peptide library member, the two domains of the GAL4 transcriptional activator protein are brought into sufficient proximity as to cause transcription of a reporter gene or a phenotypic marker gene whose expression is regulated by one or more GAL4 operators.

In another example of an internal library (see e.g., U.S. Pat. Nos. 5,270,181 and 5,292,646, both by McCoy), nucleic acid molecules encoding a multiplicity of peptides are inserted into a plasmid encoding thioredoxin such that a library of thioredoxin-peptide fusion proteins are encoded by the plasmid. The plasmid is introduced into a bacterial host cell where the thioredoxin-peptide fusion proteins are expressed cytoplasmically. The fusion proteins can be selectively released from the host cells (e.g., by osmotic shock or freeze-thaw procedures) and recovered for use in screening assays with a target.

In yet another example of an internal library (described further in Cull, M. G. et al. (1992) Proc. Natl. Acad. Sci USA 89:1865), nucleic acid molecules encoding a multiplicity of peptides are inserted into a gene encoding LacI to create a fusion gene encoding a fusion protein of LacI and the peptide library members. The plasmid encoding the fusion protein library members is designed such that the fusion proteins binds to the plasmid (i.e., a plasmid encoding the LacI fusion proteins includes lac operator sequences to which LacI binds) such that the fusion proteins and the plasmids encoding them can be physically linked. Following expression of the fusion proteins in host cells, the cells are lysed to liberate the fusion protein and associated DNA, and the library is screened with an immobilized target. Fusion proteins that bind to the target are recovered and the associated DNA is reintroduced into a cells for amplification and sequencing, thus allow for determination of the peptide sequence encoded by the DNA.

Alternative to forming a peptide library by synthesizing a multiplicity of nucleic acid molecules encoding the peptide library members, a multiplicity of peptides can be synthesized directly by standard by chemical methods known in the art. For example, a multiplicity of peptides can be synthesized by “split synthesis” of peptides on solid supports (see, e.g., Lam, K. S. et al. (1993) Bioorg. Med. Chem. Lett. 3:419424). Other exemplary chemical syntheses of peptide libraries include the pin method (see, e.g., Geysen, H. M. et al. (1984) Proc. Natl. Acad. Sci. USA 81:3998-4002); the tea-bag method (see, e.g., Houghten, R. A. et al. (1985) Proc. Natl. Acad. Sci. USA 82:5131-5135); coupling of amino acid mixtures (see, e.g., Tjoeng, F. S. et al. (1990) Int. J. Pept. Protein Res. 35:141-146; U.S. Pat. No. 5,010,175 to Rutter et al.); and synthesis of spatial arrays of compounds (see, e.g., Fodor, S. P. A. et al. (1991) Science 251:767). Peptide libraries formed by direct synthesis of the peptide library members preferably are bound to a solid support (e.g., a bead or pin, wherein each bead or pin is linked to a single peptide moiety) to facilitate separation of peptides that bind a target from peptides that do not bind a target.

A particularly preferred peptide library for use in the methods of the invention is an anchor library as described in U.S. patent application Ser. No. ______ entitled Anchor Libraries and Identification of Peptide Binding Sequences, filed on Jun. 5, 1995 (attorney docket number: P0567/7000), the entire contents of which are expressly incorporated herein by reference. As used herein, the term “anchor library” refers to a peptide library in which the peptides have non-continuous regions of random amino acids separated by specifically designated amino acid residues. Anchor libraries are therefore subsets of a complete library of a specified length. Anchor libraries can be used to identify essential contacts between a ligand and a target, and have the advantage that only a subset of all possible peptides need be synthesized and screened. In a preferred embodiment, an anchor library is made up of peptides about 16 amino acids long. An anchor library can be prepared by genetic means (e.g., by synthesizing a multiplicity of nucleic acid molecules encoding a multiplicity of anchor peptides) or by chemical means (e.g., by directly synthesizing a multiplicity of anchor peptides).

Once the peptide library has been formed, a target of interest is screened with the peptide library to identify one or more library members that bind to the target. Peptides that bind a target can be selected according to known methods, such as biopanning of an immobilized target with a phage display library. In one embodiment, a biotinylated target is immobilized on a streptavidin-coated surface either before or after contacting the target with a peptide library and unbound peptides are removed by washing. Peptide libraries bound to a solid support can be screened by, for example, contacting the peptides immobilized on the solid support with a labeled target and detecting the labeled target bound to library members or, alternatively, by releasing the peptides from the solid support and assaying the resulting solution (see, e.g., Ohlmeyer, M. H. J. et al. (1993) Proc. Natl. Acad. Sci. USA 90:10922:10926).

Following selection of one or more peptide library members that bind to the target, the amino acid sequence of the peptide is determined according to standard methods. For example, in one embodiment, the amino acid sequence of the peptide is determined by determining the nucleotide sequence of a nucleic acid molecule encoding the peptide and translating the encoded peptide using the genetic code. Nucleotide sequencing can be performed by standard methods (e.g., dideoxynucleotide sequencing or Maxam-Gilbert sequencing, either manually or using automated nucleic acid sequencers). Alternatively, in another embodiment, the amino acid sequence of the selected peptide(s) is determined by direct amino acid sequencing of the peptide (e.g., by Edman microsequencing, either manually or using automated peptide sequencers).

Once the sequence(s) of the peptide(s) that bind the target selected from the first library has been determined, a peptide motif is generated based on these sequences. As used herein, the term “peptide motif” is intended to include an amino acid consensus sequence that represents preferred amino acid residues within a peptide that are sufficient or essential for binding of the peptide to the target. Typically, the simplest way to generate a peptide motif is to compare the amino acid sequences of all peptides selected from screening a target with the first peptide library and define a peptide motif based on one or more amino acid residues that are conserved within at least two of the selected peptides. If only a single peptide is selected from the initial peptide library screening, the amino acid sequence of this peptide can constitute a peptide motif. Alternatively, when multiple peptides are selected from the initial peptide library screening, the amino acid sequences of each of the selected peptides are optimally aligned and amino acid residues conserved among two or more of the selected peptides can constitute the peptide motif. In addition to, or alternative to, direct alignment and analysis of the primary amino acid sequence of the selected peptides, a peptide motif can be generated by more sophisticated structural analysis of the selected peptides. For example, molecular modelling programs can be employed to determine structural motifs present in the selected peptide(s). Examples of such structural motifs include α-helix, β-turns, and the like (see, e.g., A. Fersht (1985) “Enzyme Structure and Mechanism”, 2nd ed., W. H. Freeman and Co., New York). Computer modelling can also be used to calculate properties of active peptides such as hydrophobicity, steric bulk, stacking interactions, dipole moment, and the like. Any of the above-mentioned properties can be included when generating a peptide motif.

In the methods of the invention, after a peptide motif has been generated for a target of interest based on screening of the first library, the target is rescreened with a second, non-peptide library that is designed based on the peptide motif. The second library can be composed of compounds that are designed to have improved properties compared to the peptides selected from screening of the first library, such as increased affinity for the target (e.g., predicted by computer modelling of the target with non-peptide compounds designed based on the peptide motif) and/or improved pharmacological properties, such as increased solubility, decreased susceptibility to proteolytic degradation, increased biodistribution and the like. Accordingly, the methods of the invention further comprise the steps of:

forming a second library comprising a multiplicity of non-peptide compounds designed based on the peptide motif;

selecting from the second library at least one non-peptide compound that binds to the target; and

determining the structure or structures of the at least one non-peptide compound that binds to the target.

The term “non-peptide compounds”, as used herein, is intended to include compounds comprising at least one molecule other than a natural amino acid residue, wherein the structures of the compounds cannot be determined by standard sequencing methodologies but rather must be determined by more complex chemical strategies, such as mass spectrometric methods. Preferred non-peptide compounds are those that, although not composed entirely of natural amino acid residues, are nevertheless related structurally to peptides, such as peptidomimetics, peptide derivatives and peptide analogues. As used herein, a “derivative” of a compound X (e.g., a peptide) refers to a form of X in which one or more reactive groups on the compound have been derivatized with a substituent group. Examples of peptide derivatives include peptides in which an amino acid side chain, the peptide backbone, or the amino- or carboxy-terminus has been derivatized (e.g, peptidic compounds with methylated amide linkages). As used herein an “analogue” of a compound X refers to a compound which retains chemical structures of X necessary for functional activity of X yet which also contains certain chemical structures which differ from X. An examples of an analogue of a naturally-occurring peptide is a peptides which includes one or more non-naturally-occurring amino acids. As used herein, a “mimetic” of a compound X refers to a compound in which chemical structures of X necessary for functional activity of X have been replaced with other chemical structures which mimic the conformation of X. Examples of peptidomimetics include peptidic compounds in which the peptide backbone is substituted with one or more benzodiazepine molecules (see e.g., James, G. L. et al. (1993) Science 260:1937-1942) and “retro-inverso” peptides (see U.S. Pat. No. 4,522,752 by Sisto), described further below.

The term mimetic, and in particular, peptidomimetic, is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ [CH₂NH], ψ[CSNH₂], ψ[NHCO], ψ[COCH₂], and ψ[(E) or (Z) CH═CH]. In the nomenclature used above, ψ indicates the absence of an amide bond. The structure that replaces the amide group is specified within the brackets. Other examples of isosteres include peptides substituted with one or more benzodiazepine molecules (see e.g., James, G. L. et al. (1993) Science 260:1937-1942), peptoids (R. J. Simon et al. (1992) Proc. Natl. Acad. Sci. USA 89:9367-9371), and the like.

Other possible modifications of peptides include an N-alkyl (or aryl) substitution (ψ [CONR]), backbone crosslinking to construct lactams and other cyclic structures, or retro-inverso amino acid incorporation (ψ[NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides.

Approaches to designing peptide analogues, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270.

The second, non-peptide library can be formed by methods known in the art for combinatorial synthesis of organic compounds. For example, a second library comprising compounds that include modified amino acids (for example, D-amino acids or synthetic amino acids such as phenylglycine) can be synthesized by techniques used for the synthesis of peptide libraries (e.g., solid support methods described supra). Other organic molecules that have been synthesized on solid supports include benzodiazepines (B. A. Bunin and J. A. A. Ellman (1992) J. Am. Chem. Soc. 114:10997-10998), peptoids (R. N. Zuckermann et al. (1992) J. Am. Chem. Soc. 114:10646-10647), peptidyl phosphonates (D. A. Campbell and J. C. Bermak (1994) J. Org. Chem. 59:658-660), vinylogous polypeptides (M. Hagihara et al. (1992) J. Am. Chem. Soc. 114:6568-6570), and the like. An alternative synthetic scheme for chemical libraries involves synthesis of compounds on resin beads wherein a coding moiety corresponding to each addition in the synthesis is also coupled to the bead (see e.g, Brenner, S. and Lerner, R. A. (1992) Proc. Natl. Acad. Sci. USA 89:5181-5183; Ohlmeyer, M. H. L. et al. (1993) Proc. Natl. Acad Sci. USA 90:10922:10926; Still et al., PCT publication WO 94/08051). In a preferred embodiment, the second library comprises compounds which include at least one peptide bond (i.e., amide bond). In a preferred embodiment, the second library is a library of peptidomimetics.

Preferably, the second library comprises at least about 10² different compounds, more preferably at least 10⁴ different compounds, and still more preferably at least 10⁶ different compounds. Depending upon the size of the non-peptide compounds in the library and the efficiency of synthesis, it may be possible to achieve a second library comprising as many as 10⁸ different compounds or even 10¹⁰ different compounds.

After formation of the second library, the target of interest is screened with the second library, e.g., by the screening methods described above for screening the first library. One or more non-peptide compounds that bind to the target are thereby selected. Preferably, a non-peptide compound selected from the second library that binds to a target has a binding affinity for the target of at least about 10⁻⁷ M, more preferably at least about 10⁻⁸ M, and even more preferably at least about 10⁻⁹ M.

Following selection of one or more compounds from the second library that bind to the target, the structure of the selected compound(s) is determined. In a preferred embodiment, the structure of the non-peptide compound(s) is determined by the use of a mass spectrometric method. Mass spectrometric methods allow for the rapid, inexpensive, and highly accurate identification of the structure of a compound based on the mass of the compound and on fragments of the compound generated in the mass spectrometer. A preferred mass spectrometric technique is tandem mass spectrometry, sometimes denoted “MS/MS”. In tandem mass spectrometry, a sample compound is first ionized and the molecular ion determined. The molecular ion is then cleaved into several smaller fragments, which are then mass-analyzed. The use of mass spectrometry to identify the structure of high-molecular weight compounds, including peptides, has been reported (see, e.g., R. S. Youngquist et al. (1995) J. Am. Chem. Soc. 117:3900; B. J. Egner et al. (1995) J. Org. Chem. 60:2652-2653). It is believed that tandem mass spectrometry is especially useful for the analysis of non-peptide compounds that containing one or more peptide bonds (e.g., peptide derivatives, peptide analogues and/or peptidomimetics) because the peptide bond can be cleaved in the spectrometer to produce fragments that can be analyzed to identify particular subunits of the compound. In certain alternative embodiments, it may be possible to analyze at least a portion of a non-peptide compound by direct amino acid sequencing, e.g., by Edman degradation (e.g., where the non-peptide compound comprises a peptide portion). Alternatively, in embodiments in which the second library is synthesized in an array (e.g., on pins or in an array on a solid surface, e.g., a “chip”), the structure of the compound can be determined by the position the compound occupies in the array. In yet other embodiments, in which the second library is an encoded library (i.e., a library in which the structure of the chemical compound has been encoded on a bead, as described in Brenner, S. and Lerner, R. A. (1992) Proc. Natl. Acad. Sci USA 89:5181-5183; Ohlmeyer, M. H. L. et al. (1993) Proc. Natl. Acad. Sci. USA 90:10922:10926; and Still et al., PCT publication WO 94/08051), the structure of the compound can be determined by decoding the encoding moiety.

In a particularly preferred embodiment of the method of the invention, the first (peptide) library is a phage display library, and the non-peptide compound(s) of the second library that bind to the target are analyzed by tandem mass spectrometry. In another particularly preferred embodiment of the methods of the invention, the first (peptide) library is an anchor library, and the compound(s) of the second library that bind to the target are analyzed by tandem mass spectrometry.

The skilled artisan will appreciate that the compound or compounds identified from the second library can be used as a basis for forming further libraries that can be used for further screening of the target. That is, the information gained from the screening of the second library can be used to design another motif, for example a modified peptide motif (e.g., a motif based on the structure of peptide derivatives, peptide analogues and/or peptidomimetics), and a subsequent, third library can be formed comprising compounds designed based on the motif generated from the screening of the second library. The target is then screened with the third library and active compounds identified as previously described herein. This process can be repeated until a compound with a desired binding affinity for the target is obtained.

Another aspect of the invention pertains to a compound identified by the method of the invention. In preferred embodiments, the compound is a peptidomimetic, peptide derivative or peptide analogue. Preferably, a compound identified by the method of the invention has a binding affinity for the target of at least about 10⁻⁷ M, more preferably at least about 10⁻⁸ M, and even more preferably at least about 10⁻⁹ M. The binding affinity of a compound for a particular target can be determined by standard methods for determining K_{d}s.

Another aspect of the invention pertains to a library comprising a multiplicity of non-peptide compounds designed based on a peptide motif, wherein the peptide motif is determined by selecting from a peptide library at least one peptide that binds to a target, determining the sequence or sequences of the at least one peptide that binds to the target and determining a peptide motif. A library of non-peptide compounds based on a peptide motif can be synthesized by the methods previously described herein. In a preferred embodiment, the non-peptide compounds of the library are peptidomimetics. Additionally or alternatively, the non-peptide compounds can be peptide derivatives and/or peptide analogues. Preferably, the library comprises at least about 10² compounds, more preferably at least about 10⁴ compounds and even more preferably at least about 10⁶ compounds. In one embodiment, the -multiplicity of non-peptide compounds are attached to a solid support, such as a plurality of resin beads.

This invention is further illustrated by the following examples which should not be construed as limiting. The contents of all references, patents and published patent applications cited throughout this application are hereby incorporated by reference.

### EXAMPLE

In this example, the method of the invention is used to identify one or more compounds that bind to a target that is expressed on the surface of a cell, the luteinizing hormone releasing hormone receptor (LHRH-R), a member of the G-protein coupled, seven transmembrane receptor superfamily.

### Construction of the First Library

A phage anchor library comprising a multiplicity of peptides is used as the first library in the method. The anchor library is comprised of peptides having random amino acid residues distributed throughout domains of alanine (Ala) and/or glycine (Gly) residues. For example, the anchor library can be composed of peptides that are sixteen amino acid residues in length and have the amino acid sequence:

X¹(Ala/Gly)₄X²(Ala/Gly)₄X³(Ala/Gly)₄X⁴

wherein X¹, X², X³and X⁴ can be any amino acid residue and each can be the same or different from the others.

To prepare the anchor library, a multiplicity of oligonucleotides encoding the peptides are synthesized by standard methods, such as the split synthesis method (See e.g., Cornack and Struhl (1993) Science 262:244-248). Synthesis of oligonucleotides for construction of anchor libraries also is described further in U.S. patent application Ser. No. ______ entitled Anchor Libraries and Identification of Peptide Binding Sequences, filed on Jun. 5, 1995 (attorney docket number: P0567/7000), the entire contents of which are expressly incorporated herein by reference.

Following synthesis, assembled oligonucleotide inserts are cloned into the pfUSE5 phage vector (Smith and Scott (1993) Methods in Enzymology 217:228-257), which allows for expression of the encoded peptides as fusions with the pIII phage coat protein. The vector (30 μg) is prepared by cleaving with 200 units of endonuclease SfiI in 500 μl of restriction buffer (Buffer #2 from New England BioLabs (NEB), Beverly, Mass.) for 10 hours. The reaction is terminated with addition of 15 mM EDTA, followed by phenol/chloroform extraction. The vector DNA is recovered by isopropanol precipitation, resuspended in 500 μl of Tris-EDTA (TE) buffer and recovered by ethanol precipitation. The phage vector is ligated to the assembled oligonucleotide inserts at 5 μg/ml vector and three-fold excess assembled insert in ligation buffer (NEB) with 100 units of T4 DNA ligase at 10°C. for 16 hours. DNA is purified from the ligation buffer by phenol/chloroform extractions, followed by ethanol precipitations and resuspension in TE buffer.

DNA from the ligation reaction is transformed into electrocompetent MC 1061 bacterial host cells (Wertman et al. (1986) Gene 49:253-262) using 0.5 μg of DNA per 100 μl of cells using 0.2 cm electroporator cells and a BioRad electroporator set at 25 μF, 2.5 KV and 200 ohms. Shocked cells are recovered in SOC media, grown out at 37°C. for 20 minutes and inoculated into LB broth containing 20 μg/ml tetracycline.

Library phage released from the transformed bacterial host cells are isolated after growing the bacterial cells for 16 hours. Phage are separated from cells by centrifugation at 4° C. at 4.2 K rpm for 30 minutes in a Beckman J6 centrifuge, followed by a second centrifugation of the supernatant at 4.2K rpm for 30 minutes. Phage are precipitated with the addition of 150 ml of 16.7% polyethyleneglycol (PEG)/3.3 M NaCl per liter of supernatant. Mixed solutions are incubated at 4° C. for 16 hours. Precipitated phage are collected by centrifugation at 4.2K rpm in a J6 centrifuge, followed by resuspension in 40 ml of Tris-buffered saline (TBS). Resuspended phage are precipitated again with the addition of 4.5 ml of PEG solution for 4 hours. Phage are collected at 5K rpm in a Beckman JA20 centrifuge at 4°C. Phage are suspended in 7 ml of TBS and brought to 1.3 mg/ml density by the addition of 1 gm of CsCl per 2.226 gm of aqueous solution. Phage are subjected to equilibrium centrifugation in a type 80 rotor at 45K rpm for 40 hours. Phage bands are isolated, diluted 20-fold with TBS and pelleted at 40K rpm in a type 50 rotor. Pellets are resuspended in 0.7 ml of TBS and as is in screening assays, described below, at approximately 3×10¹³ phage/ml.

### Screening of the First Library

To identify members of the phage anchor library that bind to LHRH-R, monolayers of cells expressing LHRH-R (such as CHO, COS or SF9 cells transfected to express LHRH-R) adhered to culture dishes are biopanned with the phage library. The phage (in TBS) are incubated with the cells for 1 hour at 4° C. and non-specific phage are removed by washing the cell monolayer with PBS containing 2% milk or 1% BSA or 10% serum for a total of 7 washes over 30 minutes. The remaining phage that are bound to the cells (by way of binding to LHRH-R on the surface of the cells) are recovered by elution with 100 μM glycine, pH 2.2 for 10 minutes. Eluted phage are neutralized with 1 M Tris base.

Eluted phage are amplified by infection of log phase K91 E. coli (Lyons and Zinder (1972) Virology 49:45-60; Smith and Scott (1993) Methods in Enzymology 217:228-257). Approximately 10⁵ phage are amplified by infecting an equal volume of K91 cells with phage at 22° C. for 10 minutes. Infected cells are diluted into 1 ml of LB broth for 30 minutes at 37° C., followed by an additional dilution with 9 ml of LB containing 20 μg/ml tetracycline and grown overnight. Phage are then separated from cells by centrifugation and purified by PEG precipitation and resuspended at 10¹² phage/ml.

To further enrich for peptides that specifically bind to LHRH-R, amplified phage can be subjected to two additional rounds of biopanning using different cell types expressing LHRH-R in each round of panning and using the binding and amplification conditions described above.

### Generation of a Peptide Motif

After biopanning, individual phage are isolated and sequenced to reveal the DNA sequence that encodes for the displayed peptide in each selected phage. Sequencing is performed by standard methods (e.g., dideoxy sequencing using Sequenase 2.0, Unites States Biochemical Co., Cleveland Ohio, according to the manufacturer's protocol).

After obtaining the DNA sequences encoding the selected peptides, the DNA sequences are optimally aligned to generate a peptide motif. The pep tide motif is determined from the amino acid residues that are conserved in at least two of the selected peptides. For example, if biopanning of the anchor library leads to selection of four peptides having the following amino acid sequences (standard three-letter abbreviations are used for amino acids):

Ser-(Ala/Gly)₄-Arg-(Ala/Gly)₄-Leu-(Ala/Gly)₄-Met

Ser-(Ala/Gly)₄-Lys-(Ala/Gly)₄-Leu-(Ala/Gly)₄-Gln

Phe-(Ala/Gly)₄-Arg-(Ala/Gly)₄-Leu-(Ala/Gly)₄-Thr

Ser-(Ala/Gly)₄-Asn-(Ala/Gly)₄-Leu-(Ala/Gly)₄-Ile

a peptide motif can be generated having the amino acid sequence:

Ser-(Ala/Gly)₄-Arg-(Ala/Gly)₄-Leu-(Ala/Gly)₄-Xaa

(wherein Xaa can be any amino acid residue).

### Construction of a Second Library

Based on the peptide motif generated from screening the target with the first library, a second library comprising a multiplicity of non-peptide compounds is synthesized by standard chemical synthesis methods (see e.g., Youngquist, R. S. et al. (1995) J. Am. Chem. Soc. 117:3900-3906; Till, J. H. et al. (1994) J. Biol. Chem. 269:7423-7428; Berman, J. et al. (1992) J. Biol. Chem. 267:1434-1437). The non-peptide compounds of the library are designed to mimic the peptide motif. For example, to create a non-peptide library based on the peptide motif described above, amino acid derivatives, analogues or mimetics of Ser at position 1, Arg at position 6, Leu at position 11 and/or Xaa at position 16 can be incorporated into the library. Derivatives, analogues and/or mimetics of the repeating Ala/Gly structure can also be incorporated into the library.

One example of a second library synthesized based on the above-described peptide motif is an analog library in which the serine at position 1 of the motif is substituted with homoserine, cyanoalanine, isoglutamine or isoasparagine, the arginine at position 6 of the motif is substituted with citrulline, isopropyllysine, homoarginine, ornithine, homocitrulline, diaminoproprionic acid, aminobenzoic acid or nitroarginine, the leucine at position 11 of the motif is substituted with NorLeu, BuGlycine, cyclohexylalanine, norval, aminobutyrl or various N-methyl aliphatic amino acids and the Xaa at position 16 of the motif is combinatorially derived from the twenty natural amino acids or standard analogs thereof.

Another example of a second library synthesized based on the above-described peptide motif is a library constructed to probe the stereochemical specificity of compounds that bind to the target by alternating D- and L-amino acids in the library. In this case, the library is constructed using the following L-amino acids: Glu, Arg, Asn, Thr, Val, Pro, Met, Tyr and His; and the following D-amino acids: Asp, Lys, Gln, Ser, Cha, Ala, Phe and Trp. The library also contains glycine. This library can define the role of D or L stereochemistry within the selected peptide motif.

Yet another example of a second library synthesized based on the above-described peptide motif is a mimetic library, wherein reduced amide mimetics are incorporated into the compounds of the library via the use of appropriate amino acid aldehyde precursors and the solid phase reductive amination procedure for assembly (Sasaki and Coy (1987) Peptides 8:119-120). Mimetics can be incorporated at one site or multiple sites within the library. Appropriate positions include sites within a peptide motif containing an aliphatic or aromatic residue, such as the leucine at position 11 of the above-described peptide motif.

Once synthesized, the library is dissolved in 1-5% dimethylsulfoxide (DMSO) in water and used in screening assays as described below.

### Screening of the Second Library

To identify members of the second library that bind to LHRH-R, membranes of CHO cells that have been transfected to express LHRH-R on their surface are prepared. One liter quantities of CHO-LHRH-R cells (e.g., 10⁹ cells/liter) are grown and harvested. The cells are lysed with a nitrogen bomb (see e.g., Autuori, F. et al. (1982) J. Cell Sci. 57:1-13). 25 ml of a washed cell suspension (in an isomolar Hanks balanced salt solution/20 mM HEPES buffer, pH 7.4) is placed in a nitrogen bomb at 4° C. with continuous stirring by a magnetic stir bar, and the pressure is adjusted to 4-500 psi, followed by continuous stirring for 20 minutes. Pressure is released into a 50 ml plastic centrifuge tube containing a 100× cocktail of protease inhibitors (0.5 mM PMSF, 10 μg/ml benzamidine, 1 μg/ml leupeptin, final concentrations). The homogenate is centrifuged for 1 hour at 5,000 ×g. The supernatant is subjected to ultracentrifugation at 50,000 ×g for one hour. The final pellet is resuspended to a concentration of about 1 mg/ml and aliquots are frozen in liquid nitrogen until used in binding assays, at which time the aliquots are thawed.

Binding reactions are set up in 12×75 mm polypropylene test tubes containing a sample of the second library (final concentration of 10 mg/ml), binding buffer (final concentrations: 10 mM Tris, 0.05% bovine serum albumin, pH 7.4) and a sample of the cell membrane preparation (approximately 10⁷ cell equivalents per tube) in a total volume of 500 μl. The binding reaction is incubated on ice for 90 minutes. The binding reaction is terminated by fast filtration binding of the mixture using a 12-well cell harvester (Millipore, Milford, Mass.). Filters (Whatman glass-fiber filters GF/C) are prewashed three times with 300 μl of 10 mM HEPES, 0.01% sodium azide. 3 ml of HEPES buffer is added to each binding reaction tube and the contents of the tube are poured over the filter in the fast filtration binding apparatus. Two additional aliquots of buffer are added to each tube and poured over the filter. Compounds from the second library that bind to the LHRH-R membrane preparation are retained on the filter, whereas compound that do not bind to the LHRH-R membrane preparation are removed.

### Identification of Compounds that Bind the Target

Compounds from the second library that bind to the LHRH-R membrane preparation are recovered from the filter of the fast filtration binding apparatus. The structures of the selected compounds are determined by tandem mass spectrometry (see e.g., Hunt, D. F., et al. (1985) Anal. Chem. 57:765-768; Hunt, D. F. et al. (1986) Proc. Natl. Acad. Sci. USA 83:6233-6237; Hunt, D. F. et al. (1987) Proc. Natl. Acad. Sci. USA 84:620-623; Biemann, K. (1990) Methods in Enzymology 193:455-479; Arnott, D. et al. (1993) Clin. Chem. 39:2005-2010; Metzger, J. W. et al. (1994) Anal. Biochem. 219:261-277; Brummel, C. L. et al. (1994) Science 264:399-402).

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for identifying a compound that binds to a target, the method comprising:
a) forming a first library comprising a multiplicity of peptides;
b) selecting from the first library at least one peptide that binds to the target;
c) determining the sequence or sequences of the at least one peptide that binds to the target, thereby generating a peptide motif;
d) forming a second library comprising a multiplicity of non-peptide compounds designed based on the peptide motif;
e) selecting from the second library at least one non-peptide compound that binds to the target; and
f) determining the structure or structures of the at least one non-peptide compound that binds to the target;
thereby identifying a compound that binds to the target.

2. The method of claim 1, wherein the first library is a phage display library.

3. The method of claim 1, wherein the first library is bound to a solid-support.

4. The method of claim 1, wherein the first library is an anchor library.

5. The method of claim 1, wherein the first library comprises at least about 10⁶ peptides.

6. The method of claim 1, wherein the first library comprises at least about 10⁹ peptides.

7. The method of claim 1, wherein the first library comprises at least about 10¹² peptides.

8. The method of claim 1, wherein step c) comprises determining the nucleotide sequence of a nucleic acid molecule or molecules that encode the at least one peptide.

9. The method of claim 1, wherein step c) comprises determining the amino acid sequence or sequences of the at least one peptide.

10. The method of claim 1, wherein the second library comprises at least one peptide derivative.

11. The method of claim 1, wherein the second library comprises at least one peptide analogue.

12. The method of claim 1, wherein the second library comprises at least one peptidomimetic.

13. The method of claim 1, wherein the second library comprises at least about 10² non-peptide compounds.

14. The method of claim 1, wherein the second library comprises at least about 10⁴ non-peptide compounds.

15. The method of claim 1, wherein the second library comprises at least about 10⁶ non-peptide compounds.

16. The method of claim 1, wherein step f) comprises analyzing the at least one non-peptide compound by a mass spectrometric method.

17. The method of claim 16, wherein the mass spectrometric method comprises tandem mass spectrometry.

18. The method of claim 1, wherein the compound that binds to a target has a binding affinity for the target of at least about 10⁻⁷ M.

19. The method of claim 1, wherein the compound that binds to a target has a binding affinity for the target of at least about 10⁻⁸ M.

20. The method of claim 1, wherein the compound that binds to a target has a binding affinity for the target of at least about 10⁻⁹ M.

21. The method of claim 1, further comprising:
g) forming a third library comprising a multiplicity of non-peptide compounds designed based on the structure or structures of the non-peptide compound or compounds determined in step f);
h) selecting from the third library at least one non-peptide compound that binds to the target; and
i) determining the structure or structures of the at least one non-peptide compound selected in step h);
thereby identifying a compound that binds to the target.

22. A method for identifying a compound that binds to a target, the method comprising:
a) forming a first library comprising a multiplicity of peptides displayed on the surface of a bacteriophage;
b) selecting from the first library at least one peptide that binds to the target;
c) determining the sequence or sequences of the at least one peptide that binds to the target, thereby generating a peptide motif;
d) forming a second library comprising a multiplicity of non-peptide compounds designed based on the peptide motif;
e) selecting from the second library at least one non-peptide compound that binds to the target; and
f) determining the structure or structures of the at least one non-peptide compound that binds to the target by tandem mass spectrometry;
thereby identifying a compound that binds to the target.

23. A method for identifying a compound that binds to a target, the method comprising:
a) forming a first library comprising an anchor library of a multiplicity of peptides;
b) selecting from the first library at least one peptide that binds to the target;
c) determining the sequence or sequences of the at least one peptide that binds to the target, thereby generating a peptide motif;
d) forming a second library comprising a multiplicity of non-peptide compounds designed based on the peptide motif;
e) selecting from the second library at least one non-peptide compound that binds to the target; and
f) determining the structure or structures of the at least one non-peptide compound that binds to the target by tandem mass spectrometry;
thereby identifying a compound that binds to the target.

24. A compound identified by the method of claim 1.

25. The compound of claim 24, which is a peptidomimetic.

26. The compound of claim 24, which binds to the target with a binding affinity of at least about 10⁻⁷ M.

27. The compound of claim 24, which binds to the target with a binding affinity of at least about 10⁻⁸ M.

28. The compound of claim 24, which binds to the target with a binding affinity of at least about 10⁻⁹ M.

29. A library comprising a multiplicity of non-peptide compounds designed based on a peptide motif, wherein the peptide motif is determined by selecting from a peptide library at least one peptide that binds to a target, determining the sequence or sequences of the at least one peptide that binds to the target and determining a peptide motif.

30. The library of claim 29, wherein library comprises at least one peptidomimetic.

31. The library of claim 29, wherein the library comprises at least about 10² non-peptide compounds.

32. The library of claim 29, wherein the library comprises at least about 10⁴ non-peptide compounds.

33. The library of claim 29, wherein the library comprises at least about 10⁶ non-peptide compounds.

34. The library of claim 29, wherein the multiplicity of non-peptide compounds are attached to a solid support.
